(19) United States
(12) Patent Application Publication
(10) Pub. No.: US 2026/0193681 A1
(43) Pub. Date: Jul. 9, 2026

(54) METHODS FOR EXTRACTING LIPID FROM YEAST

(71) Applicant: Xylome Corporation, Madison, WI (US)

(72) Inventors: Joanna D. Bundus, Madison, WI (US); Thomas W. Jeffries, Madison, WI (US); Thomas J. Kelleher, Thousand Oaks, CA (US); José Miguel Laplaza, Lansing, MI (US); Robert Mejia, Madison, WI (US); Adeline R. Zimmer, Baraboo, WI (US)

(73) Assignee: Xylome Corporation, Madison, WI (US)

(21) Appl. No.: 19/442,027

(22) Filed: Jan. 7, 2026

Related U.S. Application Data
(60) Provisional application No. 63/742,500, filed on Jan. 7, 2025.

(51) Int. Cl. C12P 7/64

(52) U.S. Cl. CPC C12P 7/64

(57) ABSTRACT

Methods of extracting lipid from yeast that do not require use of organic solvents. The methods can include releasing the yeast lipid from lipid bodies in intact yeast cells and/or lipid bodies isolated from the intact yeast cells into an aqueous solvent to provide an aqueous lipid mixture; heating the aqueous lipid mixture to a first temperature, such as a temperature 35° C. or higher; cooling the aqueous lipid mixture from the first temperature to a second temperature that is effective to aggregate the yeast lipid as lipid aggregates in the aqueous lipid mixture; and then separating the lipid aggregates from the aqueous solvent.

### CROSS-REFERENCE TO RELATED APPLICATIONS

Priority is hereby claimed to U.S. Provisional Application 63/742,500, filed Jan. 7, 2025, which is incorporated herein by reference in its entirety.

### FIELD OF THE INVENTION

The invention is directed to methods for extracting lipid from yeast, such as methods that do not require the use of organic solvents.

### BACKGROUND

The quest for sustainable and renewable energy sources has become a critical focus of global scientific and industrial efforts. This is driven by the urgent need to reduce greenhouse gas emissions, decrease reliance on fossil fuels, and minimize environmental impact. Microbial fermentation has emerged as a promising method for producing bio-oils, among various alternative energy strategies.

Microbial or single-cell oils (SCOs) are lipids produced by certain microorganisms under specific conditions. These oils have garnered significant attention due to their potential as feedstock for biodiesel production and their application in the food, pharmaceutical, and cosmetic industries. Various oleaginous microorganisms, including yeasts, fungi, and algae, can produce these oils. Some of the most commonly studied organisms include Lipomyces starkeyi, Yarrowia lipolytica, Rhodosporidium toruloides, Cryptococcus curvatus, and various strains of Chlorella and Schizochytrium. These microorganisms are recognized for accumulating substantial quantities of lipids, particularly under nutrient-limited conditions. Among them, Lipomyces starkeyi stands out as one of the most promising for this purpose due to its robust ability to produce high amounts of lipids under nutrient stress. The oils produced by Lipomyces starkeyi possess a unique composition of fatty acids, including high levels of saturated fatty acids, which makes them particularly valuable as a replacement for palm oil in cosmetics and food applications. Other organisms, such as Yarrowia lipolytica and Rhodosporidium toruloides, are also notable for producing lipids with various fatty acid profiles suitable for biodiesel and other industrial applications but are not a direct replacement for palm oil.

A strain of Lipomyces starkeyi with a fatty acid composition even closer to that of palm oil has been developed, making the oil produced by this strain being a more direct substitute for palm oil in both food and cosmetic industries (Jeffries et al. 2023). This engineered strain also boasts significantly higher productivity and lipid yield than wild-type Lipomyces starkeyi, thus increasing its commercial viability. By optimizing the metabolic pathways involved in lipid biosynthesis and accumulation, the strain produces more oil in less time and with greater efficiency, representing a significant advancement in microbial oil production.

Despite these advancements, the commercialization of microbial oils has been hindered by challenges related to extraction and purification. Conventional extraction methods typically involve using organic solvents such as hexane, chloroform (Dalmas Neto et al. 2020), or methanol. While effective at extracting lipids, these organic solvent-based methods have several significant drawbacks, including the high costs associated with organic solvent procurement, recycling, and disposal, potential health and safety risks due to organic solvent toxicity and flammability, and environmental concerns linked to organic solvent emissions and waste generation. Additionally, consumers often see organic solvent-based processes as undesirable, particularly in sectors like food, cosmetics, and nutraceuticals, where there is a growing demand for cleaner, organic solvent-free products.

Recognizing these challenges, there is a growing impetus to develop alternative, more sustainable purification methods that eliminate or significantly reduce the use of organic solvents. Organic solvent-free processes for purifying the oil generated by Lipomyces starkeyi and other yeast fermentation processes are needed.

### SUMMARY OF THE INVENTION

One aspect of the invention is directed to organic solvent-free lipid purification processes. The organic solvent-free purification processes outlined herein represent a significant technological advancement in microbial oil production. These methods eliminate organic solvent residues, making them particularly suitable for high-value food and nutraceutical applications where purity and safety are paramount. Consumers seeking environmentally friendly and health-conscious products increasingly prefer an organic solvent-free approach, adding further value to this innovation.

Moreover, the organic solvent-free nature of the processes of the invention aligns with the principles of green chemistry and sustainable industrial practices. It offers a viable pathway to meet the growing consumer demand for environmentally friendly products. The methods of the invention can lead to substantial cost savings by reducing the need for expensive organic solvents and distillation equipment while minimizing waste disposal costs. Additionally, the reduced environmental footprint enhances the overall sustainability of the microbial oil production process, making them more attractive to industries looking to reduce their carbon footprint and comply with increasingly stringent environmental regulations.

Some aspects of the invention are directed to methods of extracting lipid from yeast. In some versions, the methods comprise: providing intact yeast cells comprising lipid bodies comprising yeast lipid; releasing the yeast lipid from the lipid bodies into a first aqueous solvent to provide a first aqueous lipid mixture; heating the first aqueous lipid mixture to a first temperature, wherein the first temperature is 35° C. or higher; cooling the first aqueous lipid mixture from the first temperature to a second temperature, wherein the second temperature is a temperature effective to aggregate the yeast lipid as first lipid aggregates in the first aqueous lipid mixture; and then separating the first lipid aggregates from the first aqueous solvent.

In some versions, none of pentane, hexane, heptane, chloroform, methanol, ether, diethyl ether, methyl-tert-butyl ether (MTBE), propanol, dichloromethane, benzene, toluene, acetonitrile, ethyl acetate, and xylene is in contact with the yeast lipid during or between any of the providing the intact yeast cells, the releasing the yeast lipid from the lipid bodies into the first aqueous solvent, the heating the first aqueous lipid mixture to the first temperature, the cooling the first aqueous lipid mixture from the first temperature to the second temperature, and the separating the first lipid aggregates from the first aqueous solvent. In some versions, no organic solvent except for one or more alcohols other than methanol is in contact with the yeast lipid during or between any of the providing the intact yeast cells, the releasing the yeast lipid from the lipid bodies into the first aqueous solvent, the heating the first aqueous lipid mixture to the first temperature, the cooling the first aqueous lipid mixture from the first temperature to the second temperature, and the separating the first lipid aggregates from the first aqueous solvent.

In some versions, the first temperature is 50° C. or higher.

In some versions, the second temperature is 25° C. or lower.

In some versions, prior to separating the first lipid aggregates from the first aqueous solvent, the first aqueous lipid mixture is at least temporarily present at a first pH, wherein the first pH is at least 8.5. In some versions, the first pH is from 9 to 10.5.

In some versions, after the first aqueous lipid mixture is present at the first pH and prior to separating the first lipid aggregates from the first aqueous solvent, the first aqueous lipid mixture is adjusted from the first pH to a second pH, wherein the second pH is lower than the first pH. In some versions, the first aqueous mixture is adjusted from the first pH to the second pH prior to heating the first aqueous lipid mixture to the first temperature. In some versions, the second pH is from 6 to 8.

In some versions, the first aqueous solvent comprises a culture broth used to grow the intact yeast cells.

In some versions, the releasing the yeast lipid from the lipid bodies into the first aqueous solvent comprises simultaneously disrupting the intact yeast cells and the lipid bodies to release the yeast lipid into the first aqueous solvent. In some versions, the simultaneously disrupting the intact yeast cells and the lipid bodies comprises mechanically disrupting the intact yeast cells and the lipid bodies. In some versions, the mechanically disrupting comprises pressure homogenization.

Some versions further comprise, prior to releasing the yeast lipid from the lipid bodies into the first aqueous solvent, releasing the lipid bodies from the intact yeast cells. In some versions, releasing the lipid bodies from the intact yeast cells comprises chemically and/or enzymatically disrupting the intact yeast cells. In some versions, releasing the lipid bodies from the intact yeast cells comprises acid treating the intact yeast cells.

Some versions further comprise, after releasing the lipid bodies from the intact yeast cells, mechanically disrupting the lipid bodies to release the yeast lipid from the lipid bodies into the first aqueous solvent. In some versions, mechanically disrupting the lipid bodies comprises pressure homogenizing the lipid bodies. In some versions, the mechanically disrupting the lipid bodies comprises pressure homogenizing the lipid bodies at a pressure less than a pressure effective to break the intact yeast cells. In some versions, the mechanically disrupting the lipid bodies comprises pressure homogenizing the lipid bodies at a pressure of 12.5 kpsi or less. In some versions, the mechanically disrupting the lipid bodies comprises pressure homogenizing the lipid bodies at a pressure of 11 kpsi or less. In some versions, the mechanically disrupting the lipid bodies comprises pressure homogenizing the lipid bodies at a pressure of 5 kpsi or less.

Some versions further comprise, after separating the first lipid aggregates from the first aqueous solvent, washing the first lipid aggregates. In some versions, washing the first lipid aggregates comprises: melting the first lipid aggregates to generate melted yeast lipid; contacting the melted yeast lipid with a second aqueous solvent to generate a second aqueous lipid mixture; cooling the second aqueous lipid mixture to a temperature effective to aggregate the melted yeast lipid as second lipid aggregates; and separating the second lipid aggregates from the second aqueous solvent.

In some versions, none of pentane, hexane, heptane, chloroform, methanol, ether, diethyl ether, methyl-tert-butyl ether (MTBE), propanol, dichloromethane, benzene, toluene, acetonitrile, ethyl acetate, and xylene is in contact with the yeast lipid during or between any of the providing the intact yeast cells, the releasing the yeast lipid from the lipid bodies into the first aqueous solvent, the heating the first aqueous lipid mixture to the first temperature, the cooling the first aqueous lipid mixture from the first temperature to the second temperature, the separating the first lipid aggregates from the first aqueous solvent, and the washing the first lipid aggregates. In some versions, no organic solvent except for one or more alcohols other than methanol is in contact with the yeast lipid during or between any of the providing the intact yeast cells, the releasing the yeast lipid from the lipid bodies into the first aqueous solvent, the heating the first aqueous lipid mixture to the first temperature, the cooling the first aqueous lipid mixture from the first temperature to the second temperature, the separating the first lipid aggregates from the first aqueous solvent, and the washing the first lipid aggregates.

In some versions, the intact yeast cells are lipogenic yeast cells. In some versions, the intact yeast cells are Lipomyces yeast cells. In some versions, the intact yeast cells are Lipomyces starkeyi cells. In some versions, the intact yeast cells are genetically modified.

The objects and advantages of the invention will appear more fully from the following detailed description of the preferred embodiment of the invention made in conjunction with the accompanying drawings.

### BRIEF DESCRIPTION OF THE DRAWINGS

FIG. 1 shows a first exemplary embodiment of the invention. The process in this embodiment starts with fermentation broth. A high-pressure homogenizer (or a bead mill) breaks the yeast cells. The broth is heated (e.g., to about 35-50° C. or higher, such as about 50° C.) and then slowly cooled (e.g., to about 20-34° C. or lower, such as about 20° C.) with shaking. Aggregates of lipid form and float, and they are removed from the broth. The resulting lipid aggregates are melted and washed with water.

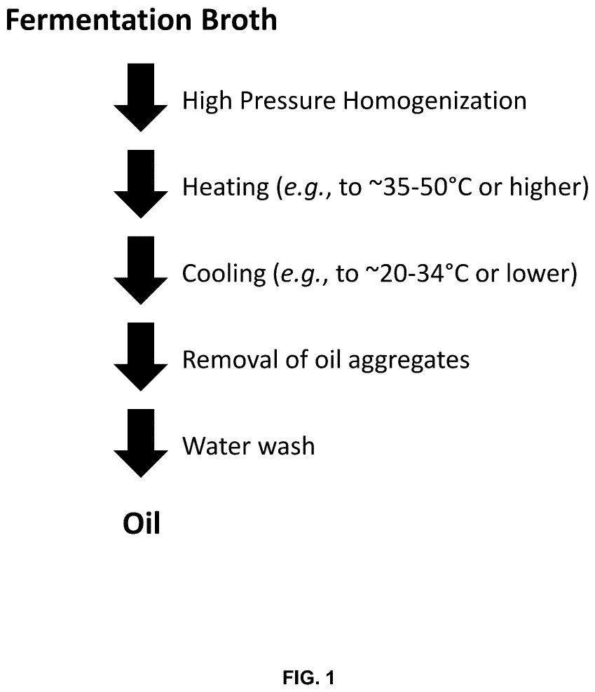

FIG. 2 shows a second exemplary embodiment of the invention. In this embodiment, the yeast cell wall is acid-hydrolyzed to release intracellular lipid bodies, and the lipid bodies are washed with water and then reisolated by flotation, filtration or centrifugation. The lipid bodies are pressure homogenized at a lower pressure than that required to break intact yeast cells. The resulting broth is heated (e.g., to about 35-50° C. or higher) and then slowly cooled (e.g., to about 20-34° C. or lower) with shaking. Aggregates of lipid form, and they are removed from the broth (e.g., by flotation, filtration, or centrifugation). The resulting lipid aggregates are melted and washed with water.

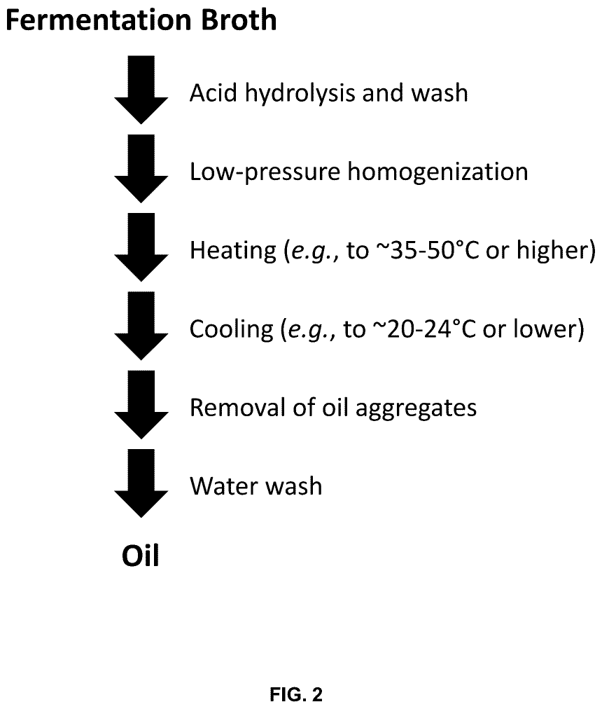

### DETAILED DESCRIPTION OF THE INVENTION

The invention is generally directed to methods of extracting lipid from yeast.

The methods of the invention can comprise providing intact yeast cells. “Intact” in this context means the yeast cells have a cell well and plasma membrane surrounding yeast organelles, lipids, and other yeast contents that are present when the yeast is alive. The term “intact,” however does not require that the yeast cells are alive, i.e., capable of further growth.

The yeast cells can comprise any type of yeast. In preferred versions, the yeast cells are lipogenic yeast. Lipogenic yeasts (also known as oleaginous yeasts) are yeasts that accumulate lipids in intracellular lipid bodies to greater than 20% of their dry mass. In some yeasts, lipids have been reported to account for up to 71% of the cell's total biomass. Out of the 1200 to 1500 known yeast species, only a fraction qualifies as lipogenic. Lipomyces starkeyi was among the earliest lipogenic yeasts studied. Other known lipogenic yeasts include Yarrowia lipolytica, and species in the genera of Rhodotorula, Cryptococcus, Candida, Trichosporon, Rhodosporidium, Sporidiobolus, Sporodobolomyces, and various other ascomyceteous and basidiomycete genera. Some lipogenic yeasts belong to the larger taxonomic groups of filamentous ascomyceteous and basidiomycetous fungi. Exemplary lipogenic yeasts include yeasts from the genus Lipomyces, such as L. starkeyi, L. anomalus, L. arxii, L. chichibuensis, L. doorenjongii, L. japonicus, L. kockii, L. kononenkoae, L. lipofer, L. mesembrius, L. oligophaga, L. orientalis, L. smithiae, L. spencermartinsiae, L. suomiensis, L. tetrasporus, L. yamadae, L. yarrowii, and L. Sp.; yeasts from the genus Yarrowia, such as Y. lipolytica, Y. bubula, Y. deformans, Y. divulgata, Y. keelungensis, Y. porcina, Y. yakushimensis, and Y. Sp.; yeasts from the genus Candida, such as C. Sp.; yeasts from the genus Hansenula, such as H. polymorpha; yeasts from the genus Cunninghamella, such as S. bigelovii sp nov CGMCC 8094, S. echinulate, S. blakesleeana JSK2, and S. Sp. Salicorn 5; yeasts from the genus Mortierella, such as M. alpina, M. isabellina, and M. Sp.; yeasts from the genus Rhodosporidium, such as R. toruloides, R. babjevae, R. diobovatum, R. fluviale, R. kratochvilovae, R. paludigenum, R. sphaerocarpum, R. araucariae, R. colostri, R. dairenensis, R. graminis, R. lusitaniae, and R. mucilaginosa; yeasts from the genus Sporidiobolus, such as S. johnsonii, S. pararoseus, S. ruineniae, S. ruineniae, and S. salmonicolor; yeasts from the genus Sporobolomyces, such as S. bannaensis, S. beijingensis, S. carnicolor, S. metaroseus, S. odoratus, S. poonsookiae, S. singularis, and S. inositophilus; yeasts from the genus Occultifur, such as O. externus; yeasts from the genus Rhodotorula, such as R. bogoriensis, R. hylophila, R. glutinis, and R. rhodochrous; yeasts from the genus Trichosporon, such as T. fermentans, T. oleaginosus ATCC 20509, and T. cutaneum; and yeasts from the genus Cryptococcus, such as C. curvatus and C. Sp.

The intact yeast can be genetically modified to increase lipid production. Various exemplary genetic modifications for increasing lipid production that can be comprised by the yeasts of the invention are described in US 2018/0245109 A1, U.S. Pat. No. 10,662,448, US 2020/0270651 A1, U.S. Pat. No. 11,168,340, US 2022/0401349 A1, and/or Jeffries et al. 2023, all of which are incorporated herein by reference. Specific exemplary modifications can include one or more recombinant diacylglycerol acyltransferase genes (such as DGA1 and/or DGA2 from Lipomyces starkeyi or homologs thereof), one or more recombinant malic enzyme genes (such as ME from Lipomyces starkeyi or homologs thereof), and/or one or more recombinant glycerol-3-phosphate acyltransferase genes (such as SCT1 from Lipomyces starkeyi or homologs thereof), all of which are described in US 2018/0245109 A1, U.S. Pat. No. 10,662,448, US 2020/0270651 A1, U.S. Pat. No. 11,168,340, US 2022/0401349 A1, and/or Jeffries et al. 2023.

The yeasts of the invention preferably comprise intracellular yeast lipid in intracellular lipid bodies. Lipid body” as used herein refers to a structure comprising lipid surrounded by a coating comprising non-lipid components. The coating can comprise phospholipid, protein, and/or other materials such as polysaccharide. The coating can be hydrophilic. The lipid bodies can have any property or characteristic as disclosed in US 2022/0401349 A1, which is incorporated herein by reference.

In some versions, the intact yeast cells are provided in a culture broth. “Culture broth” as used herein refers to a medium comprising the yeast cells in which the yeast therein can be, is being, or was grown. In some embodiments, the culture broth comprises intact, live yeast and further comprises additional components necessary and sufficient for growth of the yeast therein. In some embodiments, the culture broth is a “spent” culture broth, in which one or more components necessary and sufficient for growth of the yeast has been consumed to a level that is insufficient to support further growth. In some embodiments, the culture broth is a fermentation broth. “Fermentation broth” is a culture broth suitable for fermentation of the yeast, or a spent version of such a broth.

In some versions, providing the intact yeast cells comprises growing the yeast cells in the culture broth. In some versions, the provided intact yeast cells are processed in downstream steps (e.g., releasing the lipid bodies from the yeast cells or releasing the yeast lipid from the intact yeast cells and/or lipid bodies) without separating the yeast cells from the culture broth, e.g., by pelleting and resuspending the yeast.

After the intact yeast cells are provided, the methods of the invention can comprise a step of releasing the yeast lipid from the lipid bodies into an aqueous solvent to provide an aqueous lipid mixture. “Aqueous solvent” as used herein means a solvent comprising at least 50% v/v water, such as at least 55% v/v, at least 60% v/v, at least 65% v/v, at least 70% v/v, at least 75% v/v, at least 80% v/v, at least 85% v/v, at least 90% v/v, at least 95% v/v, or at least 99% v/v water. In addition to water, the aqueous solvent can comprise other components, such as salts, buffers, etc. In some versions, the aqueous solvent is a culture medium, such as a culture medium in which the intact yeast cells are, are being, or were grown. “Release” in this context means that the lipid body coating is at least partially or completely disrupted such that the internal lipid can be exposed to the aqueous solvent to be in contact with same. The aqueous solvent into which yeast lipid from lipid bodies is released is optionally referred to herein as the “first aqueous solvent.” Similarly, the aqueous lipid mixture formed by releasing the yeast lipid from the lipid bodies into the first aqueous solvent is optionally referred to herein as the “first aqueous lipid mixture.”

In some versions, the step of releasing the yeast lipid from the lipid bodies into the aqueous solvent comprises simultaneously disrupting the intact yeast cells and the lipid bodies to release the yeast lipid into the aqueous solvent. “Simultaneously” in this context means that both the intact yeast cells and the lipid bodies are disrupted to release their contents within a single, uninterrupted processing step and does not require that both disruptions occur exactly at the same instant within the processing step itself. For example, a given processing step can first break open the intact cells to release the lipid bodies and then subsequently break open the lipid bodies once released. Both disruptions would be considered to be “simultaneous,” however, insofar as they both occur within the same, uninterrupted processing step.

The processes employed for simultaneously disrupting the intact yeast cells and lipid bodies can comprise mechanically, chemically, and/or enzymatically disrupting the yeast cells. The disruption in such a case would be sufficient to disrupt both the intact cells and the lipid bodies comprised thereby. Exemplary methods of the invention comprise mechanically disrupting the intact yeast cells and lipid bodies. Methods of mechanical disruption are well known in the art. These include but are not limited to beating, grinding, and/or shearing and can comprise vortexing, beat beating, and/or pressure-drop homogenization. Vortexing can comprise adding binding beads to a sample in a tube and repeatedly vortexing the tube. Bead beating can comprise rapidly agitating a sample with grinding media (beads or balls) in a bead beater (device that shakes the homogenization vessel). The beads or balls employed in vortexing and bead beading are typically made of glass (silica), ceramic (zirconium), or steel. Pressure homogenization is a method that comprises exerting greater than atmospheric pressure on a sample to force it through an orifice. A combination of sheer force and acceleration force exerted on the cells in the sample as they are forced through the orifice can break open the cells and release their inner contents. For example, a sample can be momentarily trapped in a high-pressure cylinder comprising one or more orifices. A set pressure greater than ambient can be exerted on the sample, forcing the sample to enter the orifice. Shear stress can be exerted as the sample enters an orifice, and an acceleration stress can be exerted as the sample accelerates in the orifice. A number of pressure homogenizers are known in the art, including those disclosed in the following examples.

In some versions, the aqueous solvent is a culture medium for the intact cells, such that simultaneously disrupting the intact yeast cells and the lipid bodies releasees the yeast lipid directly into the culture medium. In some versions, simultaneously disrupting the intact yeast cells and the lipid bodies to release the yeast lipid into the aqueous solvent, such as the culture medium, is preferred for minimizing processing steps for increased efficiency.

Some versions of the invention comprise releasing the lipid bodies from the intact yeast cells prior to releasing the yeast lipid from the lipid bodies into the first aqueous solvent. “Prior to” in this context refers to releasing the lipid bodies from the intact yeast cells in a separate, previous processing step than that used to release the yeast lipid from the lipid bodies into the first aqueous solvent. Once released from the intact yeast cells, the released lipid bodies can be separated from the medium into which they are released (in some versions, for example, the culture medium) and optionally washed. The separation can comprise floating the lipid bodies in the medium, centrifugation, or filtration, among other methods. The washing can comprise mixing the lipid bodies in an aqueous wash solvent and reisolating them. The lipid bodies can be washed more than once, such as 2 times, 3 times, 4 times, or more, before conducting further processing steps. After isolating the lipid bodies from the medium and/or the last wash solvent, the lipid bodies can be mixed with the aqueous solvent into which the yeast lipid contained therein is ultimately released. In some versions, releasing the lipid bodies from the intact yeast cells prior to releasing the yeast lipid from the lipid bodies into the first aqueous solvent is preferred for obtaining a cleaner, purer lipid preparation, as the lipid bodies can be separated from cellular debris, culture medium components, and/or other potential contaminants before the yeast lipid is released from the lipid bodies.

The lipid bodies can be released from the intact yeast cells through mechanically, chemically, and/or enzymatically disrupting the intact yeast cells in a manner that does not break the lipid bodies. Exemplary methods for releasing lipid bodies from intact yeast cells are described in US 2022/0401349, which is incorporated herein by reference.

In some versions, intact lipid bodies are released from the intact cells by chemically disrupting the intact yeast cells. The chemically disrupting can comprise acid treating the lipid bodies. The acid treating can comprise adding an acid to the lipid bodies. The acid can comprise a strong acid. Exemplary acids include H₂SO₄ (sulfuric acid), HNO₃ (nitric acid), HBr (hydrobromic acid), HClO₄ (perchloric acid), and HI (hydroiodic acid), among others. The acid treatment can be performed with heating for various lengths of time. The heating can include heating to temperatures from about 50° C. to about 140° C. or more. The lengths of time can range from about 30 minutes to about 18 hours. In some versions, the heating can comprise autoclaving. The autoclaving can be performed at about 121° C. for about 45 minutes. The acid used to hydrolyze the cells can include sulfuric acid, phosphoric acid, oxalic acid, or citric acid, among others, or any combination thereof. The acid treatment can involve mixing intact cell suspensions with volumes of the acid. The mixing can include mixing the cell suspensions in 1- to 10-fold volumes of an acid solution. The acid solution in some versions includes the acid in an amount from about 0.1 N to about 3 N. Lipid bodies can be recovered by mixing cell suspensions in 1- to 10-fold volumes of 0.25 to 0.5 N sulfuric acid followed by autoclaving at 121° C. for 45 minutes. Exemplary release methods include mixing yeast cell suspensions with about 0.25 N sulfuric acid, about 0.5 N phosphoric acid, about 0.4 N oxalic acid, or about 2 N citric acid at about 50° to about 121° C. for various lengths of time ranging from about 30 min up to about 18 h. The time, temperature, and acidity can be varied to attain hydrolysis of the cell walls and release lipid bodies. Other chemical methods for releasing lipid bodies from intact cells is described in US 2022/0401349, which is incorporated herein by reference.

In some versions, the lipid bodies are released from the intact yeast cells by enzymatically disrupting the yeast cells. Exemplary enzymes that can be used in the enzymatic digestion include yeast lytic enzymes. Exemplary yeast lytic enzymes include β-(1,3)-glucanases, such as those from EC 3.2.1.6 and 3.2.1.39. β-(1,3)-glucanases comprising a Ricin B Lectin binding domain are preferred, such as β-(1,3)-glucanases found in Cellulosimicrobium cellulans and Trichoderma reesei. Other exemplary enzymes are described in the following examples and in US 2022/0401349, which is incorporated herein by reference.

In some versions, the lipid bodies are released from the intact yeast cells by mechanically disrupting the yeast cells. Exemplary mechanical methods include bead beating (e.g., using a “bead beater” apparatus with titanium beads) and sonicating. Exemplary mechanical methods are described in US 2022/0401349, which is incorporated herein by reference.

After releasing the lipid bodies from the intact cells and the optional washing, the lipid bodies can be processed to release internal yeast lipid therefrom into the aqueous solvent. The disruption can occur to an extent that at least some of the yeast lipid contacts the aqueous solvent. In some versions, disrupting the lipid bodies in the lipid-body mixture can be performed by mechanically disrupting the intact lipid bodies. The mechanical disruption of the lipid bodies can be performed with any method of mechanical disruption described herein, including but not limited to beating, grinding, and/or shearing with vortexing, beat beating, and/or pressure homogenization.

In some versions, the mechanically disrupting the lipid bodies comprises pressure homogenizing the lipid bodies with a pressure homogenizer. In some versions, the pressure homogenizing is performed at a pressure less than a pressure effective to break the intact yeast cells. “A pressure effective to break the intact yeast cells” as used herein refers to a pressure equivalent to break the intact yeast cells from which the lipid bodies were released, as it is not expected that intact yeast cells would be present at the step in which the released intact lipid bodies are disrupted. In various versions of the invention, the lipid bodies are pressure homogenized at a pressure of 12.5 kpsi or less, such as 12 kpsi or less, 11.5 kpsi or less, 10 kpsi or less, 9.5 kpsi or less, 9 kpsi or less, 8.5 kpsi or less, 8 kpsi or less, 7.5 kpsi or less, 7 kpsi or less, 6.5 kpsi or less, 6 kpsi or less, 5.5 kpsi or less, or 5 kpsi or less. In various versions of the invention, the lipid bodies are pressure homogenized at a pressure of 0.1 kpsi or more, 0.5 kpsi or more, 1 kpsi or more, 1.5 kpsi or more, 2 kpsi or more, 2.5 kpsi or more, 3 kpsi or more, 3.5 kpsi or more, 4 kpsi or more, 4.5 kpsi or more, or 5 kpsi or more.

After generating the aqueous lipid mixture comprising the yeast lipid in contact with the aqueous solvent (either directly from the yeast cells or from separately isolated lipid bodies), the aqueous lipid mixture can then be heated to a first temperature. The first temperature can be a temperature of 30° C. or higher, 35° C. or higher, 40° C. or higher, 45° C. or higher, 50° C. or higher, 55° C. or higher, or, in some instances, at temperatures that result in steam. The first temperature is preferably less than 150° C., such as 145° C. or less, 140° C. or less, 135° C. or less, 130° C. or less, 125° C. or less, 120° C. or less, 115° C. or less, 110° C. or less, 100° C. or less, 95° C. or less, 90° C. or less, 85° C. or less, 80° C. or less, 75° C. or less, 70° C. or less, 65° C. or less, or 60° C. or less.

The aqueous lipid mixture can then be cooled from the first temperature to a second temperature. The second temperature can be a temperature effective to form lipid aggregates from the yeast lipid in the aqueous lipid mixture. In various versions of the invention, the second temperature is 34° C. or lower, such as 33° C. or lower, 32° C. or lower, 31° C. or lower, 30° C. or lower, 29° C. or lower, 28° C. or lower, 27° C. or lower, 26° C. or lower, 25° C. or lower, 24° C. or lower, 23° C. or lower, 22° C. or lower, 21° C. or lower, 20° C. or lower, 15° C. or lower, 10° C. or lower, or 5° C. or lower. The second temperature is preferably but not necessarily greater than 0° C. The lipid aggregates formed in the first aqueous lipid mixture are optionally referred to herein as “first lipid aggregates.”

Once the lipid aggregates are formed within the aqueous lipid mixture, the aggregates can be separated from the aqueous solvent. In the process, the aggregates are also separated from elements dissolved in the aqueous solvent. In some versions, the lipid aggregates are separated from the aqueous solvent by filtering the aggregates from the liquid base. In some versions, the lipid aggregates are separated from the liquid base by centrifuging the aqueous lipid mixture to form an aggregate layer comprising the lipid aggregates and a liquid layer comprising the liquid base and separating the aggregate layer and the liquid layer. In some versions, the lipid aggregates are separated from the aqueous solvent by floating the lipid aggregates on the aqueous solvent and removing the floated lipid aggregates from the aqueous solvent.

In some versions, the aqueous lipid mixture is at least temporarily present at a first pH prior to separating the lipid aggregates from the aqueous solvent, prior to cooling the aqueous lipid solvent from the first temperature to the second temperature, prior to heating the aqueous lipid solvent to the first temperature, and/or prior to releasing the yeast lipid from the lipid bodies into the aqueous solvent to provide the aqueous lipid mixture. In some versions, for example, the aqueous solvent is present at the first pH (e.g., adjusted to have the first pH) prior to releasing the prior to releasing the yeast lipid from the lipid bodies into the aqueous solvent to provide the aqueous lipid mixture. In some versions, the aqueous lipid mixture is present at the first pH (e.g., adjusted to have the first pH) after releasing the yeast lipid from the lipid bodies into the aqueous solvent, such as immediately after releasing the yeast lipid from the lipid bodies into the aqueous solvent and prior to heating the aqueous lipid mixture to the first temperature. Providing the aqueous lipid mixture at least temporarily at the first pH helps to solubilize various pigments or other impurities that may be present in the aqueous lipid mixture in the aqueous solvent. This results in a purer lipid preparation when the lipid aggregates are ultimately separated from the aqueous solvent, as evidenced by a whiter color of the lipid aggregates compared to identical processes in which the aqueous lipid mixture is not at least temporarily present at the first pH. The aqueous solvent and/or the aqueous lipid mixture (depending on the embodiment) can be adjusted to the first pH using any base. An exemplary base is sodium carbonate.

In various versions of the invention, the first pH is at least 8.0, at least 8.1, at least 8.2, at least 8.3, at least 8.4, at least 8.5, at least 8.6, at least 8.7, at least 8.8, at least 8.9, at least 9.0, at least 9.1, at least 9.2, at least 9.3, at least 9.4, at least 9.5, at least 9.6, at least 9.7, at least 9.8, at least 9.9, at least 10.0, at least 10.1, at least 10.2, at least 10.3, at least 10.4, or at least 10.5. In various versions of the invention, the first pH is no more than 10.0, no more than 10.1, no more than 10.2, no more than 10.3, no more than 10.4, no more than 10.5, no more than 10.6, no more than 10.7, no more than 10.8, no more than 10.9, no more than 11.0, no more than 11.1, no more than 11.2, no more than 11.3, no more than 11.4, no more than 11.5, no more than 11.6, no more than 11.7, no more than 11.8, no more than 11.9, or no more than 12. In some versions, the first pH is from 9 to 10.5.

In some versions of the invention, the aqueous lipid mixture is adjusted from the first pH to a second pH prior to separating the lipid aggregates from the aqueous solvent, prior to cooling the aqueous lipid solvent from the first temperature to the second temperature, prior to heating the aqueous lipid solvent to the first temperature, and/or prior to releasing the yeast lipid from the lipid bodies into the aqueous solvent to provide the aqueous lipid mixture. In some versions, the aqueous lipid mixture is adjusted from the first pH to a second pH prior to heating the aqueous lipid mixture to the first temperature. The second pH is lower than the first pH. Adjusting the aqueous lipid mixture from the first pH to the second pH increases formation and separation of the lipid aggregates from the aqueous solvent. The aqueous lipid mixture can be adjusted from the first pH to the second pH using any suitable acids. Exemplary acids include citric acid.

In various versions of the invention, the second pH is at least at least 5.5, at least 5.6, at least 5.7, at least 5.8, at least 5.9, at least 6.0, at least 6.1, at least 6.2, at least 6.3, at least 6.4, at least 6.5, at least 6.6, at least 6.7, at least 6.8, at least 6.9, at least 7.0, at least 7.1, at least 7.2, at least 7.3, at least 7.4, or at least 7.5. In various versions of the invention, the second pH is 7.5, no more than 7.6, no more than 7.7, no more than 7.8, no more than 7.9, no more than 8.0, no more than 8.1, no more than 8.2, no more than 8.3, no more than 8.4, no more than 8.5, no more than 8.6, no more than 8.7, no more than 8.8, no more than 8.9, or no more than 9.0. In some versions, the second pH is from 6 to 8.

In some versions of the invention, a salt, such as a metal salt, is added to the aqueous lipid mixture prior to separating the lipid aggregates from the aqueous solvent, prior to cooling the aqueous lipid solvent from the first temperature to the second temperature, prior to heating the aqueous lipid solvent to the first temperature, and/or prior to releasing the yeast lipid from the lipid bodies into the aqueous solvent to provide the aqueous lipid mixture. Addition of the metal salt helps with the formation and/or separation of the lipid aggregates from the aqueous solvent. In various versions, the salt is added in an amount of at least 2.0% w/v, at least 2.5%, w/v, at least 3.0% w/v, at least 3.5% w/v, at least 4.0% w/v, at least 4.5% w/v, or at least 5.0% w/v of the added salt in the aqueous lipid mixture. In various versions, the salt is added in an amount of up to 4.5% w/v, up to 5.0% w/v, up to 5.5% w/v, up to 6.0% w/v, up to 6.5% w/v, up to 7.0% w/v, up to 7.5% w/v, up to 8.0% w/v, up to 8.5% w/v, up to 9.0% w/v, up to 9.5% w/v, or up to 10.0% w/v of the added salt in the aqueous lipid mixture. “Added salt” in this context refers only to the salt added to the aqueous lipid mixture and does not account for the concentration of any salts already in the aqueous lipid mixture prior to the addition of the added salt. Exemplary metals in the metal salts include lithium, sodium, potassium, magnesium, calcium, or other Group 1 or Group 2 metals. Exemplary salts include sodium sulfate and sodium citrate.

After separating the first lipid aggregates from the first aqueous solvent, the first lipid aggregates can be washed with an aqueous solvent. Washing can be performed by melting the first aggregates in a second aqueous solvent such as water or buffered water (e.g., heated to the first temperature as described above) then cooling (e.g., to the second temperature as described above or in a refrigerator at 3 to 8° C.) until the melted lipid aggregates into second lipid aggregates that form a layer that floats on top of the second aqueous solvent. Alternatively, the second aqueous solvent can be added to the melted lipid aggregates after melting. The second aqueous solvent can be removed either by perforating the surface and drawing it off or by draining it from the bottom, among other methods, including centrifugation and filtration. Generally, the separated lipid can be washed one or more times in the same manner. In a final step, the melted lipid can be removed by centrifugation and decantation. The aqueous solvents with which the first lipid aggregates are washed are optionally referred to herein as the “second aqueous solvent” to distinguish them from the “first aqueous solvent,” the latter of which is the aqueous solvent into which the yeast lipid from the lipid bodies is released. Similarly, the lipid aggregates formed in the washing steps are optionally referred to herein as “second lipid aggregates” to distinguish them from the first lipid aggregates, the latter of which are the lipid aggregates formed in the first aqueous solvent to form the first aqueous lipid mixture. The first aqueous solvent and second aqueous solvents can have the same or different compositions. Similarly, each second aqueous solvent (if more than one wash is performed) can have the same or different compositions. The temperatures for the melting and cooling in the washing can be the same as those described above for heating and cooling to form the initial aggregates in the initial aqueous solution (e.g., the first and second temperatures). Similarly, the separation steps in the washing steps can be performed with the same methods as those described above for separating the initially generated lipid aggregates from the initial aqueous solution.

In preferred versions of the invention, lipid extraction steps provided herein are performed without the use of organic solvents typically used in the process of purifying lipids. In some versions, for example, the lipid extraction steps are performed such that the yeast lipid (whether in aggregate form (e.g., the first lipid aggregates in the initial isolation or the second lipid aggregates generated in the washing) or liquid form) does not contact select organic solvents during or between such steps. Exemplary extraction steps during or between which the yeast lipid is not contacted with the select organic solvents can include any one or more of the providing the intact yeast cells comprising lipid bodies comprising yeast lipid, the releasing the yeast lipid from the lipid bodies into the first aqueous solvent to provide the first aqueous lipid mixture, the heating the first aqueous lipid mixture to the first temperature, the cooling the first aqueous lipid mixture from the first temperature to the second temperature, the separating the first lipid aggregates from the first aqueous solvent, and the washing the first lipid aggregates, or any combination of steps otherwise described herein. In some versions, the select organic solvents excluded from the extraction steps include pentane, hexane, heptane, chloroform, methanol, ether, diethyl ether, methyl-tert-butyl ether (MTBE), propanol, dichloromethane, benzene, toluene, acetonitrile, ethyl acetate, xylene, and any combination thereof. In some versions, the select organic solvents excluded from the extraction steps include one or more organic solvents provided in Table 1, in any combination.

TABLE 1
Exemplary organic solvents.

| | | Solubility (g solvent/ |
|---|---|---|
| Solvent | Formula | 100 g water) |
| acetic acid | C₂H₄O₂ | Miscible |
| acetone | C₃H₆O | Miscible |
| acetonitrile | C₂H₃N | Miscible |
| benzene | C₆H₆ | 0.18 |
| 1-butanol | C₄H₁₀O | 6.3 |
| 2-butanol | C₄H₁₀O | 15 |
| 2-butanone | C₄H₈O | 25.6 |
| t-butyl alcohol | C₄H₁₀O | Miscible |
| carbon tetrachloride | CCl₄ | 0.08 |
| chlorobenzene | C₆H₅Cl | 0.05 |
| chloroform | CHCl₃ | 0.795 |
| cyclohexane | C₆H₁₂ | 0.0055 |
| 1,2-dichloroethane | C₂H₄Cl₂ | 0.861 |
| diethylene glycol | C₄H₁₀O₃ | 10 |
| diethyl ether | C₄H₁₀O | 7.5 |
| diglyme (diethylene glycol | C₆H₁₄O₃ | Miscible |
| dimethyl ether) | | |
| 1,2-dimethoxy- | C₄H₁₀O₂ | Miscible |
| ethane (glyme, DME) | | |
| dimethyl- | C₃H₇NO | Miscible |
| formamide (DMF) | | |
| dimethyl sulfoxide (DMSO) | C₂H₆OS | Miscible |
| 1,4-dioxane | C₄H₈O₂ | Miscible |
| ethanol | C₂H₆O | Miscible |
| ethyl acetate | C₄H₈O₂ | 8.7 |
| ethylene glycol | C₂H₆O₂ | Miscible |
| glycerin | C₃H₈O₃ | Miscible |
| heptane | C₇H₁₆ | 0.01 |
| hexamethylphosphoramide | C₆H₁₈N₃OP | Miscible |
| (HMPA) | | |
| hexamethylphosphorous | C₆H₁₈N₃P | Miscible |
| triamide (HMPT) | | |
| hexane | C₆H₁₄ | 0.0014 |
| methanol | CH₄O | Miscible |
| methyl t-butyl | C₅H₁₂O | 5.1 |
| ether (MTBE) | | |
| methylene chloride | CH₂Cl₂ | 1.32 |
| N-methyl-2-pyrrolidinone | CH₅H₉NO | Miscible |
| (NMP) | | |
| nitromethane | CH₃NO₂ | 9.50 |
| pentane | C₅H₁₂ | 0.04 |
| petroleum ether (ligroine) | — | — |
| 1-propanol | C₃H₈O | Miscible |
| 2-propanol | C₃H₈O | Miscible |
| pyridine | C₅H₅N | Miscible |
| tetrahydrofuran (THF) | C₄H₈O | soluble |
| toluene | C₇H₈ | 0.05 |
| triethyl amine | C₆H₁₅N | 0.02 |
| o-xylene | C₈H₁₀ | Insoluble |
| m-xylene | C₈H₁₀ | Insoluble |
| p-xylene | C₈H₁₀ | Insoluble |

In some versions, the select organic solvents excluded from the lipid extraction steps include all organic solvents except for alcohols other than methanol. It is noted herein that reference to a general organic solvent (e.g., hexane) comprises all structural (e.g., n-hexane, isohexane, etc.) and stereoisomers thereof, unless specified otherwise.

It is understood herein that the first lipid aggregates and second lipid aggregates are both to be considered forms of the yeast lipid insofar as at least some of lipid originally contained in the lipid bodies is present in the first lipid aggregates and/or second lipid aggregates.

“Oil” as used herein refers to lipid in any physical state, whether solid or liquid, and does not necessarily imply a liquid state.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Example 1. XYL403 Fermentation Broth

XYL403 fermentation broth was made as follows. Seventy-five milliliters of YPD (10 g/L yeast extract, 20 g/L peptone, and 20 g/L dextrose) in a 500-ml baffled Erlenmeyer flask was inoculated from a single colony of freshly plated Lipomyces starkeyi strain XYL403 (Y-11557+P_{CIT1}-SCT1-T_{CIT1}+P_{TP1}-DGA2-T_{PGK1}+P_{TEF1}-DGA1-TTDH3+P_{TPI1}-ME-T_{PYK1}) (US 2022/0401349 A1, Jeffries et al. 2023) and grown at 28° C. with shaking for three days. 50 ml of the overnight was used to inoculate 1920 ml of a medium containing 25.1 grams of yeast extract FNI100 (Lallemand), 21.5 grams of (NH₄)₂SO₄, 1 gram of CaCl₂): 2H₂O, 3.5 grams of KH₂PO₄, 1.7 grams Mg₂SO₄·7H₂O, 0.2 milliliters of antifoam 204, and 2 milliliters of a 10.32 grams per liter solution of Fe(NH₄)₂(SO₄)₂ plus 160 grams of a 53% (w/w) dextrose solution. The fermentation was done in a 7 L Applikon Biotechnology fermenter. The pH was controlled at ~5.2+/−0.5 with 5 N NaOH. Air flow was set up at 2 L per minute, temperature at 28° C., and agitation at 700 revolutions per minute. Dextrose was added during fermentation to keep a concentration of around 10 to 20 grams per liter. The fermentation ran for 10 to 14 days. Cells were heat-killed by increasing the temperature to 65° C. and incubating the broth for at least 1 hour.

### Example 2. High-Pressure Homogenization (11 Kpsi) of XYL403 Fermentation Broth

High-pressure homogenized fermentation broth was generated by pressure homogenizing around 2000 grams of XYL403 fermentation broth at 11 kpsi using a Constant Systems Cell Disruptor. 512 grams of the high-pressure homogenized fermentation broth were heated to 50° C. and then placed at 24° C. overnight in a 1 L bottle. The next day, 19.1 grams of lipid aggregates were formed. The lipid aggregates were isolated from the broth by flotation. The lipid aggregates were then washed by melting them in the presence of water at 50° C., mixing with the water, reforming lipid aggregates at 24° C., and isolating the lipid aggregates. The lipid recovery yield from the high-pressure homogenized fermentation broth was 34%.

An alkalized high-pressure homogenized fermentation broth was generated by adding 24.2 grams of sodium carbonate to 1515 grams of the high-pressure homogenized fermentation broth to reach a pH of about 10. 512 grams of the alkalized high-pressure homogenized fermentation broth was heated to 50° C. and then placed at 24° C. overnight in a 1 L bottle. The next day, 35.6 grams of lipid aggregates were formed. The lipid aggregates were isolated from the broth. The lipid aggregates were then washed and reisolated from the wash as described above. The lipid recovery yield from the alkalized high-pressure homogenized fermentation broth was 54%. The lipid recovered from the alkalized high-pressure homogenized fermentation broth was whiter in color than that recovered high-pressure homogenized fermentation broth, indicating that the increase in pH helps to solubilized pigments and other contaminants in the broth and accordingly separate such components from the lipid.

A neutralized high-pressure homogenized fermentation broth was generated by adding 55 grams of sodium citrate to the 1186.1 g of the alkalized high-pressure homogenized fermentation broth and adjusting the pH to 7.2 using concentrated citric acid. 591.4 grams of the neutralized high-pressure homogenized fermentation broth were heated to 50° C. and then placed at 24° C. overnight in a 1 L bottle. The next day, 38 grams of lipid aggregates were formed. The lipid aggregates were isolated from the broth. The lipid aggregates were then washed and reisolated from the wash as described above. The lipid recovery yield from the neutralized high-pressure homogenized fermentation broth was 61%. The lipid recovered from the neutralized high-pressure homogenized fermentation broth was white in color similar to that recovered from the alkalized high-pressure homogenized fermentation broth, indicating that the neutralization of the alkalized high-pressure homogenized fermentation broth does not affect the solubilization of pigments and other contaminants in the broth. The sodium citrate acted as a buffer and added ionic strength to the broth. The increased added ionic strength and neutral pH were believed to help form the lipid aggregates to increase yield.

The 585 g of neutralized high-pressure homogenized fermentation broth was spun at 3000 g. This did not result in a lipid layer, so the broth was heated to 50° C. and then placed at 24° C. overnight in a 1 L bottle. The next day, 33 g of lipid aggregates were formed. The lipid aggregates were isolated from the broth. The lipid aggregates were then washed and reisolated from the wash as described above. The lipid recovery yield from this broth was 53%.

Experiments were conducted in which XYL403 fermentation broth was adjusted to about pH 10 prior to homogenizing at 11 kpsi, adding sodium citrate, readjusting the pH to about 7.2 with citric acid, heating to 50° C., aggregating lipid at 24° C., and washing. These experiments showed that the alkalinization of the XYL403 fermentation broth can occur before or after homogenization with similar results.

### Example 3. Effect of pH on High-Pressure Homogenized (11 kpsi) Fermentation Broth

To 500 ml of XYL403 fermentation broth, 8 grams of sodium carbonate was added to achieve a pH of 10. The cells were then disrupted with a pressure homogenizer at a pressure of 11 kpsi with a Constant Systems Cell Disruptor. The homogenized broth was split five ways, and the pH adjusted to 3.0, 4.5, 7.0, 8.5, and 9.8 with citric acid. The broth was heated to 50° C. and then placed in a 250 ml shake flask and shaken at 150 rpm 24° C. overnight. The next day, the pH 8.5 broth had the heaviest lipid aggregates, followed by 9.8, with 7.0 having moderate lipid aggregates, and pH 3.0 and 4.0 had small lipid aggregates.

### Example 4. Double High-Pressure Homogenization (15 Kpsi) and Controlled Cooling Improves Lipid Aggregation and Purification

A 3.647-Kg Sample of XYL403 Fermentation Broth (106 g/Kg of Microbial Lipid) was processed as follows. To the broth, 59 g of sodium carbonate was added to reach a pH of 10.5 and mixed until dissolved. The mixture was then subjected to two passes (2×) through a Constant Systems high-pressure homogenizer at 15 kpsi. After homogenization, 147 g of 0.5 M citric acid was added, lowering the pH to 8.4.

The resulting broth was divided equally into four 2-liter bottles. Each bottle was heated to 60° C. with shaking at 150 rpm and then allowed to cool passively to room temperature while still shaking. Within approximately two days, lipid aggregation was observed. The aggregates were collected by sieving. The remaining sieved broth (4.06 kg) contained 49.4 g/kg of residual lipid, corresponding to 200.4 g of lipid (52% of the total lipid).

The lipid aggregates were further purified by sequential washing: first and second washes with deionized water (the lipid aggregates were mixed with approximately 2 L each, heated to 60° C., mixed thoroughly, and cooled overnight at 4° C. to reaggregate the lipid), followed by two additional washes with water containing 1 mL of 0.5 M NaOH. The washed lipid contained small particulate flakes that were removed by heating the lipid to 70° C. and centrifuging at 9,000 rpm for 10 minutes. The upper lipid layer was collected, yielding 128 g of clarified lipid.

The overall purification yield was 33%. Fatty acid composition analysis revealed no significant difference from the initial broth lipid composition, indicating that the low-energy, solvent-free purification maintained the native lipid profile.

### Example 5. Production of Isolated Lipid Bodies

Lipid bodies were released from cells by adding 13.3 milliliters of 96% concentrated H₂SO₄ to 1 L of XYL403 fermentation broth and autoclaving it at 121° C. for 1 hour. The released lipid bodies rose by flotation, and the denser bottom water was removed to generate isolated lipid bodies.

### Example 6. High-Pressure Homogenization (11 Kpsi) of Isolated Lipid Bodies

Isolated lipid bodies were made as described in Example 5 from five liters of XYL403 fermentation broth, containing 83.4 grams of lipid per kg of broth. The isolated lipid bodies were washed three times by allowing the lipid bodies to settle on top, draining around 80% of the volume and replacing it with double deionized water. The washed lipid bodies were resuspended in water in a final volume of 5 liters. pH was raised to 10.3 using 16 g/L of sodium carbonate.

Two and a half liters of the above solution were pressure homogenized at 11 kpsi with a Constant Systems Cell Disruptor to generate an 11-kpsi-homogenized lipid body solution. The lipid concentration in the 11-kpsi homogenized lipid body solution was 80.5 g/kg, as determined below using the methods in Example 9.

The pH of a 500 ml aliquot of the 11-kpsi-homogenized lipid body solution was adjusted to 7 with concentrated sulfuric acid. 500 ml of isohexane was added and mixed for at least 45 min at 50° C. The solution was centrifuged at 13,700×g. The isohexane phase was removed. The isohexane was evaporated, and 39 grams of lipid was recovered for a yield of 97%.

The pH of a 500 ml aliquot of 11-kpsi-homogenized lipid body solution was adjusted to 7 with concentrated sulfuric acid. 500 ml of isohexane and 500 ml of ethanol were added and mixed for at least 45 min at 50° C. The solution was centrifuged at 13,700×g. The isohexane phase was removed. The isohexane was evaporated, and 36 grams of lipid was recovered for an 89% yield.

The pH of a 500 ml aliquot of 11-kpsi-homogenized lipid body solution was adjusted to 7 with concentrated sulfuric acid. The pH-adjusted aliquot was heated to 70° C. for 10 min and spun at 13,700×g. A lipid layer was observed, but no clear separation was obtained.

The pH of a 500 ml aliquot of the 11-kpsi-homogenized lipid body solution was adjusted to 8 with concentrated sulfuric acid. The solution was placed in a one-liter bottle. After heating the solution to 50° C., the bottle was left to cool to room temperature (~20° C.) with gentle shaking (~150 rpm). Lipid aggregates formed and were removed from the solution. The concentration of the lipid remaining in the solution was 55.1 g/kg and the lipid recovered was 25.4 g for a recovery yield of 63%.

### Example 7. Low-Pressure Homogenization (5 Kpsi) of Lipid Bodies

Isolated lipid bodies were made as described in Example 5 from five liters of XYL403 fermentation broth, containing 83.4 grams of lipid per kg of broth. The isolated lipid bodies were washed three times by allowing the lipid bodies to settle on top, draining around 80% of the volume and replacing it with double deionized water. The washed lipid bodies were resuspended in a final volume of 5 liters. pH was raised to 10.3 using 16 g/L of sodium carbonate.

Two and a half liters of the above solution were pressure homogenized at 5-kpsi with a Constant Systems Cell Disruptor to generate a 5-kpsi homogenized lipid body solution. The lipid concentration in the 5-kpsi-homogenized lipid body solution was 74.6 g/kg, as determined below in Example 9.

The pH of a 500 ml aliquot of the 5-kpsi-homogenized lipid body solution was adjusted to 7 using concentrated sulfuric acid. 500 ml of isohexane was added and mixed for at least 45 min at 50° C. The solution was centrifuged at 13,700×g. The isohexane phase was removed. The isohexane was evaporated, and 33.9 g of lipid was recovered, which yielded 91%.

The pH of a 500 ml aliquot of the 5-kpsi-homogenized lipid body solution was adjusted to 7 using concentrated sulfuric acid. 500 ml of isohexane and 500 ml of ethanol were added and mixed for at least 45 min at 50° C. The solution was centrifuged at 13,700×g. The isohexane phase was removed. The isohexane was evaporated, and 35.3 g of lipid was recovered for a yield of 95%.

The pH of a 500 ml aliquot of 5-kpsi-homogenized lipid body solution was adjusted to 7 with concentrated sulfuric acid. The pH-adjusted aliquot was heated to 70° C. for 10 min and spun at 13,700×g. A lipid layer was observed, but no clear separation was obtained.

The pH of a 500 ml aliquot of the 5-kpsi-homogenized lipid body solution was adjusted to 8 using concentrated sulfuric acid. The solution was placed in a one-liter bottle. After heating the solution to 50° C., the bottle was left to cool to room temperature (~20° C.) with gentle shaking (~150 rpm). Lipid aggregates formed and were removed from the solution. The concentration of the lipid remaining in the solution was 55.1 g/kg.

### Example 8. Isolation of Lipid Bodies Using Yeast Cell Wall Degrading Enzyme from Oerskovia xantineolytica

The present example shows the isolation of lipid bodies using yeast cell well degrading enzyme from Oerskovia xantineolytica. This method can be used as an alternative to isolating lipid bodies with the acid treatment as described above in Example 5.

Oerskovia xanthineolytica enzyme solution was prepared as follows. 25 ml of Autolyzed Yeast medium (AY) (in 1.0 L H₂O: 1.6 g autolyzed yeast; 8.0 g FNI 100 primary yeast extract (Lallemand, Inc.); 4.0 g glucose; 1.0 g MgSO₄·H₂O; 0.2 g CaCl₂·2H₂O; 26 mg Fe(NH₄)₂SO₄; pH to 6.9 with 28 ml of 1 M K₂HPO₄) was added to a 125-ml flask and inoculated with a loop of Oerskovia xanthineolytica (Cellulosimicrobium cellulans) NRRL β-16088 (ATCC 27402). The inoculated AY was placed on shaker at 150 rpm at 30° C. to generate an O. xantineolytica culture. 125 ml of the O. xantineolytica culture was used to inoculate 900 ml of AY medium in a bioreactor and incubated at 28° C., pH 6.47, with 250 rpm of agitation for 24 h to generate O. xantineolytica cell broth, which is a yeast cell wall lytic enzyme preparation. The O. xantineolytica cell broth (900 ml) was divided into three centrifuge bottles and cells were harvested by centrifugation at 8,000 rpm for 20 min. From these three batches, approximately 835 ml of supernatant solution containing O. xantineolytica enzyme (“O. xantineolytica enzyme solution”) was decanted into a sterile 1.0-L bottle. The pH of O. xantineolytica enzyme solution was 7.35.

L. starkeyi XYL403 was cultivated in a bioreactor; 500 ml of L. starkeyi broth was removed, and the pH was adjusted to 4.4.

Yeast cell lytic reactions were set up in duplicate:
1. 40 ml L. starkeyi cell broth+10 ml of O. xantineolytica enzyme solution;
   pH=4.69; adjusted to pH 7.0 with KOH.
2. 40 ml L. starkeyi cell broth+10 ml of O. xantineolytica enzyme solution;
   pH=4.6; no pH adjustment.
3. 40 ml L. starkeyi cell broth+10 ml of water (control); pH=3.87; adjusted to pH 7.0 with KOH.
   All three reactions were placed on a shaker at 50° C. for 18 h. Reactions 1 and 2 showed cell-wall degradation and released lipid body-like structures. Comparisons of enzymatic reactions at 50° C. and 37° C. showed that 50° C. was more effective.

### Example 9. Quantification of Lipid

The quantification of lipid in the foregoing examples was performed using gravimetric analysis such as the following as an exemplary method. In a 15-ml conical centrifuge tube, 2 ml of sample and 0.4 ml of concentrated 38% HCl are added. The tube is placed in a boiling water bath for 1 hour with occasional mixing. Once cool, 6 ml of Folch's Solvent (2:1 chloroform:methanol v/v) is added. The mixture is vortexed for 5 minutes at room temperature, and then 3 ml of 1 M NaCl solution is added. The tube is then centrifuged at 3,000 rpm for 5 minutes. The lower chloroform phase is removed using a glass Pasteur pipette and placed in a pre-weighed, flat-bottom aluminum dish. The chloroform is evaporated, and the remaining lipid is measured.

### REFERENCES

Dalmas Neto, C. J., Sydney, E. B., Candeo, E. S. et al. New Method for the Extraction of Single-Cell Oils from Wet Oleaginous Microbial Biomass: Efficiency, Oil Characterisation and Energy Assessment. Waste Biomass Valorization, 2020. 11(7): 3443-3452.
Jeffries, T. W., Kelleher, T. J. and Mokry, D. Z, Precision Fermentation of Bioidentical Palm Oil Alternatives. Cosmetics and Toiletries Peer Reviewed Journal, 2023. 138 (3): 34-37 and DM15-DM17.
Pomraning K R, Bredeweg E L, Baker S E. Regulation of Nitrogen Metabolism by GATA Zinc Finger Transcription Factors in Yarrowia lipolytica. mSphere. 2017 Feb. 15; 2(1):e00038-17.

1. A method of extracting lipid from yeast comprising:
providing intact yeast cells comprising lipid bodies comprising yeast lipid;
releasing the yeast lipid from the lipid bodies into a first aqueous solvent to provide a first aqueous lipid mixture;
heating the first aqueous lipid mixture to a first temperature, wherein the first temperature is 35° C. or higher;
cooling the first aqueous lipid mixture from the first temperature to a second temperature, wherein the second temperature is a temperature effective to aggregate the yeast lipid as first lipid aggregates in the first aqueous lipid mixture; and then
separating the first lipid aggregates from the first aqueous solvent.

2. The method of claim 1, wherein none of pentane, hexane, heptane, chloroform, methanol, ether, diethyl ether, methyl-tert-butyl ether (MTBE), propanol, dichloromethane, benzene, toluene, acetonitrile, ethyl acetate, and xylene is in contact with the yeast lipid during or between any of the providing the intact yeast cells, the releasing the yeast lipid from the lipid bodies into the first aqueous solvent, the heating the first aqueous lipid mixture to the first temperature, the cooling the first aqueous lipid mixture from the first temperature to the second temperature, and the separating the first lipid aggregates from the first aqueous solvent.

3. The method of claim 1, wherein no organic solvent except for one or more alcohols other than methanol is in contact with the yeast lipid during or between any of the providing the intact yeast cells, the releasing the yeast lipid from the lipid bodies into the first aqueous solvent, the heating the first aqueous lipid mixture to the first temperature, the cooling the first aqueous lipid mixture from the first temperature to the second temperature, and the separating the first lipid aggregates from the first aqueous solvent.

4. The method of claim 1, wherein the first temperature is 50° C. or higher, wherein the second temperature is 25° C. or lower.

5. (canceled)

6. The method of claim 1, wherein prior to separating the first lipid aggregates from the first aqueous solvent, the first aqueous lipid mixture is at least temporarily present at a first pH, wherein the first pH is at least 8.5.

7. (canceled)

8. The method of claim 6, wherein, after the first aqueous lipid mixture is present at the first pH and prior to separating the first lipid aggregates from the first aqueous solvent, the first aqueous lipid mixture is adjusted from the first pH to a second pH, wherein the second pH is lower than the first pH.

9. The method of claim 8, wherein the first aqueous mixture is adjusted from the first pH to the second pH prior to heating the first aqueous lipid mixture to the first temperature.

10. The method of claim 8, wherein the second pH is from 6 to 8.

11. The method of claim 1, wherein the first aqueous solvent comprises a culture broth used to grow the intact yeast cells.

12. The method of claim 1, wherein the releasing the yeast lipid from the lipid bodies into the first aqueous solvent comprises simultaneously disrupting the intact yeast cells and the lipid bodies to release the yeast lipid into the first aqueous solvent.

13. The method of claim 12, wherein the simultaneously disrupting the intact yeast cells and the lipid bodies comprises mechanically disrupting the intact yeast cells and the lipid bodies, wherein the mechanically disrupting comprises pressure homogenization.

14. (canceled)

15. The method of claim 1, further comprising, prior to releasing the yeast lipid from the lipid bodies into the first aqueous solvent, releasing the lipid bodies from the intact yeast cells.

16. The method of claim 15, wherein the releasing the lipid bodies from the intact yeast cells comprises chemically and/or enzymatically disrupting the intact yeast cells.

17. The method of claim 15, wherein the releasing the lipid bodies from the intact yeast cells comprises acid treating the intact yeast cells.

18. The method of claim 15, further comprising, after releasing the lipid bodies from the intact yeast cells, mechanically disrupting the lipid bodies to release the yeast lipid from the lipid bodies into the first aqueous solvent.

19. The method of claim 18, wherein the mechanically disrupting the lipid bodies comprises pressure homogenizing the lipid bodies, wherein the mechanically disrupting the lipid bodies comprises pressure homogenizing the lipid bodies at a pressure less than a pressure effective to break the intact yeast cells.

20-21. (canceled)

22. The method of claim 1, further comprising, after separating the first lipid aggregates from the first aqueous solvent, washing the first lipid aggregates, wherein the washing the first lipid aggregates comprises:
melting the first lipid aggregates to generate melted yeast lipid;
contacting the melted yeast lipid with a second aqueous solvent to generate a second aqueous lipid mixture;
cooling the second aqueous lipid mixture to a temperature effective to aggregate the melted yeast lipid as second lipid aggregates; and
separating the second lipid aggregates from the second aqueous solvent.

23. The method of claim 22, wherein none of pentane, hexane, heptane, chloroform, methanol, ether, diethyl ether, methyl-tert-butyl ether (MTBE), propanol, dichloromethane, benzene, toluene, acetonitrile, ethyl acetate, and xylene is in contact with the yeast lipid during or between any of the providing the intact yeast cells, the releasing the yeast lipid from the lipid bodies into the first aqueous solvent, the heating the first aqueous lipid mixture to the first temperature, the cooling the first aqueous lipid mixture from the first temperature to the second temperature, the separating the first lipid aggregates from the first aqueous solvent, and the washing the first lipid aggregates.

24. The method of claim 22, wherein no organic solvent except for one or more alcohols other than methanol is in contact with the yeast lipid during or between any of the providing the intact yeast cells, the releasing the yeast lipid from the lipid bodies into the first aqueous solvent, the heating the first aqueous lipid mixture to the first temperature, the cooling the first aqueous lipid mixture from the first temperature to the second temperature, the separating the first lipid aggregates from the first aqueous solvent, and the washing the first lipid aggregates.

25. (canceled)

26. The method of claim 1, wherein the intact yeast cells are Lipomyces yeast cells, wherein the intact yeast cells are genetically modified.

27-28. (canceled)
